(19) United States
(12) Patent Application Publication
(10) Pub. No.: US 2008/0045727 A1
(43) Pub. Date: Feb. 21, 2008

(54) Chiral 3-Halophthalic Acid Derivatives

(76) Inventors: Harry Blaschke, Wuppertal (DE); Sergiy Pazenok, Solingen (DE)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/660,668

(22) PCT Filed: Aug. 18, 2005
(86) PCT No.: PCT/EP05/08945

§ 371 Date: Feb. 21, 2007

(30) Foreign Application Priority Data
Aug. 31, 2004 (DE) 10 2004 042 138.2
Dec. 16, 2004 (DE) 10 2004 060 502.5

(51) Int. Cl. C07D 307/77; C07C 63/00

(52) U.S. Cl. 549/303; 562/430

(57) ABSTRACT

The invention relates to new salts from chiral 3-halophthalic acid derivatives, a method for their production, their use for the production of chiral phthalic acid diamides as well as a new method for the production of chiral phthalic acid diamides.

The invention relates to new salts from chiral 3-halophthalic acid derivatives, a method for their production, their use for the production of chiral phthalic acid diamides as well as a new method for the production of chiral phthalic acid diamides.

It is known that the reaction of 3-halophthalic acid anhydrides with nucleophiles leads to isomeric mixtures whose formation is dependent on the halogen atom and on the type of nucleophile (J. Org. Chem. 1977, 42, 3425-3431). Furthermore, it is known that 3-halo-N substituted phthalic acid derivatives are suitable as intermediate products for the production of pest control agents (compare, for example, EP-A 0 919 542 and EP-A 1 006 107). 3-halo-N substituted phthalic acid derivatives are obtained by causing 3-halophthalic acid anhydrides to react with amines (compare, for example, EP-A 0 919 542 and EP-A 1 006 107). The yields from this method leave much to be desired. The undesired isomers (i.e. the 6-halo analogs) result at a certain percentage from 8 to 20%, and additional high losses of 10 to 20% result with the isolation of the desired isomer, which are the result of similar physical characteristics (for example, solubility) of both isomers. The technical use of one such method is therefore scarcely possible for economic reasons.

There was thus the problem of making available a method suitable for industrial implementation, by means of which very good yields of chiral 3-halophthalic acid derivatives can be obtained from an easily accessible source material using inexpensive auxiliary materials with justifiable energy input and avoiding the accumulation of large amounts of undesired isomers.

Surprisingly, it was then found that one obtains the salts of 3-halophthalic acid derivatives starting from 3-halophthalic acid anhydride after conversion with a chiral nucleophile and treatment with a suitable base, which can be isolated very easily in pure form. In particular, it was surprisingly found that the lithium or sodium salts of the undesired isomers remain in solution.

As a result, the losses during the isolation can be minimised and the yield and purity of the desired product increased.

The subject of the present invention is thus a method for the production of 3-halophthalic acid derivatives of the formula (I) 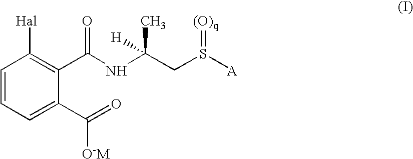
in which
Hal stands for halogen,
A stands for C₁-C₆-alkyl, C₃-C₆-alkenyl, C₃-C₆-alkinyl, C₁-C₆-alkoxy-C₁-C₄-alkyl, C₁-C₆-alkylthio-C₁-C₄-alkyl, C₁-C₆-alkylsulfinyl-C₁-C₄-alkyl, (C₁-C₆-alkyl)carbamoyl,
q stands for 0, 1 or 2,
M stands for an alkali metal ion, an earth alkali metal ion, tetra(C₁-C₄-alkyl)ammonium or tetra(C₁-C₄-alkyl)phosphonium, whereby in the case of an earth alkali metal ion respectively two molecules of a compound of the formula (I) form a salt with one such ion,
   characterised in that one causes to react
   (A) 3-halophthalic acid anhydrides of the formula (I) 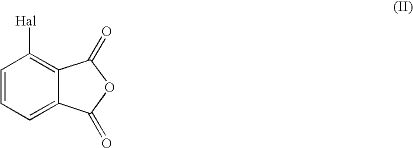
   in which Hal has the meaning indicated above,
   with amines of the formula (III) 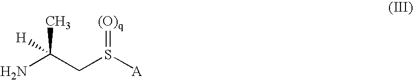
   in which A and q have the meanings indicated above,
   in the presence of a hydroxide of the formula (IV)
      M(OH⁻)ₜ (IV)
   in which
   M has the meaning indicated above,
   t stands for 1 if M stands for alkali metal ion, tetra(C₁-C₄-alkyl)ammonium or tetra(C₁-C₄-alkyl)phosphonium,
   t stands for 2 if M stands for an earth alkali metal ion.

After carrying out the conversion, the desired product can be obtained in high yield and of very good quality by precipitation.

The 3-halophthalic acid anhydrides to be used as educts in method (A) according to the invention are generally defined by the formula (II). Hal preferably stands for fluorine, chlorine, bromine or iodine in this formula (II).

The 3-halophthalic acid anhydrides of the formula (II) are known.

3-bromophthalic acid anhydride is obtained, for example, by diazotising 2,3-dimethylaniline with sodium nitrite, converting the diazonium salt with potassium bromide in 2,3-dimethylbromobenzene, and then oxidising, for example, with potassium permanganate or oxygen.

3-iodophthalic acid anhydride can be obtained in the same manner as the 3-bromophthalic acid anhydride. Alternatively, 3-iodophthalic acid anhydride is obtained by hydrogenating 3-nitrophthalic acid first to the 3-aminophthalic acid (for example, hydrogen, nickel catalyst) and then replacing the amino group with iodine in a Sandmeyer reaction.

Furthermore, the amines to be used as educts in method (A) according to the invention are generally defined by the formula (III). In this formula (III), A and q preferably stand for the following meanings:
A preferably stands for methyl, ethyl, n- or iso-propyl, n-, iso-, sec- or tert-butyl, allyl, propargyl, methoxymethyl or methylthiomethyl.
A particularly preferably stands for methyl, ethyl, n- or iso-propyl, n-, iso-, sec- or tert-butyl.
A very particularly preferably stands for methyl, ethyl or iso-propyl.
q preferably stands for 0, 1 or 2.
q also preferably stands for 1.
q particularly preferably stands for 0.
q also particularly preferably stands for 2.

Amines of the formula (III) can be obtained according to known methods (compare WO 01/23350 and WO 03/099777).

Furthermore, the hydroxides to be used as educts in method (A) according to the invention are generally defined by the formula (IV). In this formula (IV), M preferably stands for lithium, sodium, potassium, calcium, magnesium, barium, tetrabutylammonium, tetrabutylphosphonium, particularly preferably for lithium, sodium, potassium, calcium, tetrabutylammonium, tetrabutylphosphonium, very particularly preferably for lithium or sodium.

Hydroxides of the formula (IV) are known synthesis chemicals.

The method (A) according to the invention can be carried out in the presence of a suitable inert diluent. Most notably coming into consideration as diluents are: hydrocarbons such as, for example, pentane, hexane, heptane, octane, cyclohexane, methylcyclohexane, benzene, toluol, xylol, petrol ether, ligroin; halogenated hydrocarbons such as, for example, dichloromethane, trichloromethane, tetrachloromethane, 1,2-dichlorethane, chlorobenzene or dichlorobenzene; nitriles such as acetonitrile, propionitrile, butyronitrile; ethers such as, for example, diethyl ether, methyl ethyl ether, diisopropyl ether, dibutyl ether, dioxane, dimethoxyethane (DME), tetrahydrofuran (THF), diethylene glycol dimethyl ether (DGM); esters such as, for example, ethyl acetate, amyl acetate; acid amides such as, for example, dimethylformamide (DMF), dimethylacetamide (DMA), N-methylpyrrolidone, 1,3-dimethyl-2-imidazolidinone, hexamethylphosphoric acid triamide (HMPA). N-methylpyrrolidone, butyronitrile, dimethylacetamide (DMA), dioxane, 1,3-dimethyl-2-imidazolidinone are particularly preferred as diluents.

The method (A) according to the invention can be carried out within a relatively large temperature range. The conversion is preferably carried out at temperatures between −10° C. and +80° C., in particular between 0° C. and 30° C.

The method (A) according to the invention is generally carried out under standard pressure. However it is also possible to carry out the method (A) according to the invention under increased or decreased pressure—in general between 0.1 bar and 50 bar, preferably between 1 bar and 10 bar.

For carrying out the method (A) according to the invention, one generally adds between 1 mole and 1.5 moles, preferably between 1.05 moles and 1.2 moles of an amine of the formula (III) and also between 1 mole and 1.5 moles, preferably between 1.05 and 1.2 moles of a hydroxide of the formula (IV) to 1 mole of 3-halophthalic acid anhydride of the formula (II).

Furthermore, the subject matter of the present invention is a method for the production of 3-halophthalic acid derivatives of the formula (I-a) 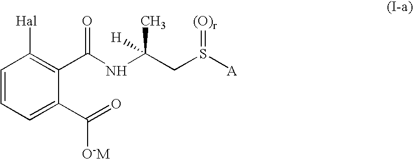
in which
r stands for 1 or 2 and
Hal, A and M have the meanings indicated above,
   characterised in that one causes to react
   (B) 3-halophthalic acid derivatives of the formula (I-b) 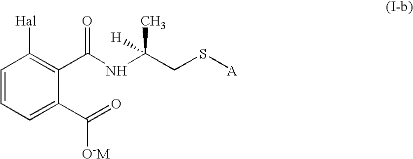
   in which Hal, A and M have the meanings indicated above,
   in the presence of an oxidising agent.

The 3-halophthalic acid derivatives required as educts for carrying out the method according to the invention are generally defined by the formula (I-b). In this formula (I-b), Hal, A and q preferably, particularly preferably, very particularly preferably or especially preferably have the meanings that were indicated above in connection with the description of the source materials for the formulas (II), (III) and (IV) for the moieties as preferred, particularly preferred, very particularly preferred or especially preferred.

3-halophthalic acid derivatives of the formula (I-b) are obtained as per the method (A) according to the invention.

All usual agents for such reactions can be used as oxidising agents for carrying out the method (B) according to the invention. Especially usable are hydrogen peroxide, peroxy acids such as, for example, peracetic acid (CH₃COOOH), trifluoroperacetic acid (CF₃COOOH), metachloroperbenzoic acid (m-ClC₆H₄COOOH), potassium permanganate or oxygen.

The following solvents are suitable as diluents for carrying out the method (B) according to the invention: Alcohols such as, for example, methanol, ethanol, iso-propanol, trifluorethanol; halogenated hydrocarbons such as, for example, dichloromethane, trichloromethane, tetrachloromethane, 1,2-dichlorethane, chlorobenzene or dichlorobenzene; nitrites such as acetonitrile, propionitrile, butyronitrile; water, 1,3-dimethyl-2-imidazolidinone, hexamethylphosphoric acid triamide (HMPA), N-methylpyrrolidone, dimethylacetamide (DMA), dioxane, 1,3-dimethyl-2-imidazolidinone, acetic acid or trifluoroacetic acid.

If the method (13) according to the invention is carried out in the presence of a base [preferably in the presence of an, alkali metal hydroxide of the formula (IV)], the 3-halophthalic acid derivatives are obtained in the form of their salts.

If the method (B) according to the invention is carried out under acidic conditions (for example with the use of the peroxy acids as an oxidising agent, see also the production examples), the free benzoic acid derivatives of the formula (I-c) (q=1, 2; see below) are obtained instead of the salts of the formula (I-a).

Furthermore, the present invention relates to new 3-halophthalic acid derivatives of the formula (I). 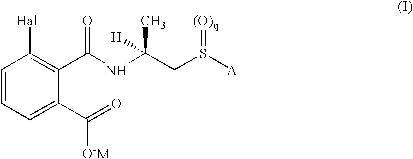
in which Hal, A, q and M have the meanings indicated above.

The 3-halophthalic acid derivatives according to the invention are generally defined by the formula (I). In this formula (I), Hal, A, q and M preferably, particularly preferably, very particularly preferably or especially preferably have the meanings that were indicated above in connection with the description of the source materials of the formulas (II), (III) and (IV) for the moieties as preferred, particularly preferred, very particularly preferred or especially preferred.

Furthermore, the present invention relates to new 3-halophthalic acid derivatives of the formula (I-c) 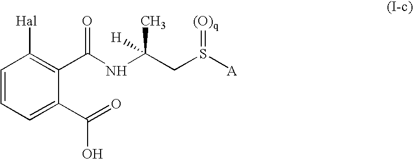
in which Hal, A and q have the meanings indicated above.

The 3-halophthalic acid derivatives according to the invention are generally defined by the formula (I-c). In this formula (I-c), Hal, A and q preferably, particularly preferably, very particularly preferably or especially preferably have the meanings that were indicated above in connection with the description of the educts of the formulas (II), (III) and (IV) for the moieties as preferred, particularly preferred, very particularly preferred or especially preferred.

The 3-halophthalic acid derivatives according to the invention of the formula (I) can be used for the production of chiral phthalic acid diamides.

Thus phthalic acid diamides of the formula (V) are obtained 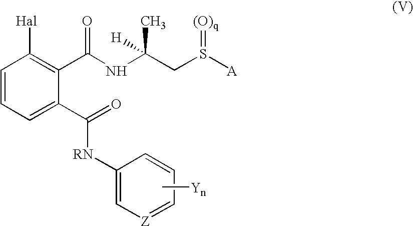
in which
Hal stands for halogen,
A stands for C₁-C₆-alkyl, C₃-C₆-alkenyl, C₃-C₆-alkinyl, C₁-C₆-alkoxy-C₁-C₄-alkyl, C₁-C₆-alkylthio-C₁-C₄-alkyl, C₁-C₆-alkylsulfinyl-C₁-C₄-alkyl, (C₁-C₆-alkyl)carbamoyl,
q stands for 0, 1 or 2,
R stands for hydrogen or C₁-C₆ alkyl,
Z stands for CY or N,
Y stands for hydrogen, halogen, C₁-C₆ alkyl, C₁-C₆-halogenoalkyl, C₁-C₆-alkoxy, C₁-C₆-halogenoalkoxy, C₁-C₆-alkylthio, C₁-C₆-halogenoalkylthio or cyano,
n stands for 0, 1, 2, 3, 4 or 5,
   by converting
   (C) 3-halophthalic acid derivatives of the formula (I) 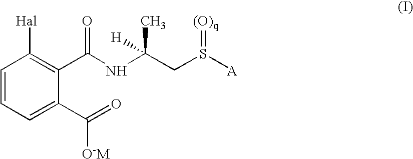
   in which Hal, A, q and M have the meanings indicated above,
   first with a dehydrating reagent into the corresponding isophthalimides of the formula (VI) 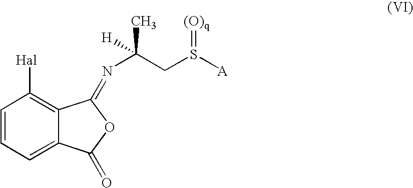
   in which Hal, A and q have the meanings indicated above,
   and causing them to react after isolation or without further isolation with arylamines of the formula (VII) 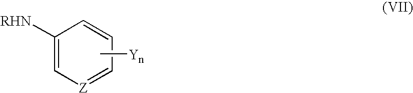
   in which R, Z, Y and n have the meanings indicated above,
   if necessary in the presence of a diluent (for example, dichloroethane) and if necessary in the presence of an acid (for example, hydrochloric acid).

Phthalic acid diamides of the formula (V), in which q stands for 0 or 1, can be converted in a simple manner into the sulfones, i.e. phthalic acid diamides of the formula (V), in which q stands for 2. Suitable as oxidising agents are, for example, hydrogen peroxide, peroxy acids such as, for example, peracetic acid (CH₃COOOH), trifluoroperacetic acid (CF₃COOOH), metachloroperbenzoic acid (m-ClC₆H₄COOOH), potassium permanganate or oxygen.

The 3-halophthalic acid derivatives of the formula (I) required as starting materials for carrying out the method (C) according to the invention were already described above in connection with the method (A) according to the invention.

The isophthalimides that are formed as an intermediate product while carrying out the method (C) according to the invention are generally defined by the formula (VI). In this formula Hal, A and q preferably, particularly preferably, very particularly preferably or especially preferably stand for the meanings that were indicated above in connection with the description of the source materials of the formulas (II), (III) and (IV) for the moieties as preferred, particularly preferred, very particularly preferred or especially preferred.

Isophthalimides of the formula (VI) are novel and likewise a subject matter of the present invention. Isophthalimides of the formula (VI) are obtained according to the first step of the method (C) according to the invention and subsequent isolation (compare also the production examples).

Furthermore, the arylamines required as starting materials for carrying out the method (C) according to the invention are generally defined by the formula (VII). In this formula, R, Z, Y and n preferably, particularly preferably or very particularly preferably have the following specified meanings.
R preferably stands for hydrogen, methyl, ethyl, n- or iso-propyl, n-, iso-, sec- or tert-butyl,
R particularly preferably stands for hydrogen, methyl, ethyl, iso-propyl n- or tert-butyl,
R very particularly preferably stands for hydrogen
z preferably stands for CY or N,
Y preferably stands for fluorine, chlorine, bromine, C₁-C₄-alkyl, C₁-C₃-halogenoalkyl, C₁-C₄-alkoxy, C₁-C₃-halogenoalkoxy, C₁-C₄-alkylthio, C₁-C₃-halogenoalkylthio or cyano,
Y furthermore preferably stands for hydrogen
Y particularly preferably stands for chlorine, methyl, ethyl, n- or iso-propyl, trifluoromethyl, pentafluoromethyl, heptafluoroisopropyl, hexafluoroisopropyl or bromohexafluoroisopropyl
Y furthermore particularly preferably stands for hydrogen n preferably stands for 0, 1, 2, 3 or 4,
n particularly preferably stands for 1, 2 or 3,
n very particularly preferably stands for 2,
n furthermore very particularly preferably stands for 1.

Arylamines of the formula (VII) are known or can be obtained in known ways (compare EP-A 0 936 212, EP-A 1 006 102, EP-A 1 418 169, EP-A 1 418 171).

Based on the method according to the invention, the cyclization resulting in the isophthalimide of the formula (VI) is carried out in the presence of a dehydrating reagent. Phosgene, thionyl chloride, POCl₃, chloroformic acid ester and trifluoroacetic acid anhydride are preferably usable. Chloroformic acid alkyl esters such as the methyl-, ethyl- or propyl esters are particularly preferably used.

The synthesis of the isophthalimide of the formula (VI) can be carried out in the presence of a base. Preferred as a base are alkali metal hydroxides or -carbonates such as, for example, sodium hydroxide, potassium hydroxide, lithium hydroxide, sodium carbonate or sodium hydrogen carbonate; amines such as, for example, triethylamine, ethyldiisopropylamine, diazabicyclooctane (DABCO), pyridine, picoline, 4-dimethylaminopyridine. Particularly preferably one uses sodium hydroxide or sodium hydrogen carbonate.

The synthesis of the isophthalimide of the formula (VI) is carried out in the presence of a diluent. Preferably included here are nitriles such as, for example, acetonitrile, propionitrile, butyronitrile; halogenated hydrocarbons such as, for example, chlorobenzene, dichlorobenzene, dichloromethane, chloroform, dichloromethane. One can use the mixture of two or more diluents or also a 2-phase system such as, for example, water/butyronitrile, water/methylene chloride, water/toluol, water/chlorobenzene.

The synthesis of the isophthalimide of the formula (VI) can also be drastically simplified and improved through the addition of phase transfer catalysts (PTC) (for example, tetramethylammonium bromide, tetrabutylammonium hydroxide, tetrabutylammonium hydrogen sulfate, tetraphenylphosphonium bromide, 18-crown-6). Thus it is possible to carry out the reaction in the presence of PTC even at room temperature, i.e. at temperature from 20-25° C., preferably 20° C., instead of at 40-50° C. In doing so, the formation of by-products can be repressed. While carrying out the method according to the invention with PTC, one employs 0.5 to 5 moles % of the catalyst for 1 mole of the salt of the formula (I) for creating the isophthalimide of the formula (VI).

The synthesis of the isophthalimide of the formula (VI) can be carried out within a relatively wide temperature range. In general, one works at temperatures from 0° C. to 80° C., preferably at temperatures of 10° C. to 60° C.

While carrying out the method (C) according to the invention, one adds 0.9 to 1.5 moles of the dehydrating reagent to 1 mole of the salt of the formula (I) for creating the isophthalimide of the formula (VI).

The conversion with the arylamines of the formula (VII) takes place in the presence of a diluent. Preferably included here are nitriles such as acetonitrile, propionitrile, butyronitrile; halogenated hydrocarbons such as, for example, chlorobenzene, dichlorobenzene, dichloromethane, chloroform, dichloromethane.

The conversion with the arylamines of the formula (VII) can be further accelerated through the addition of catalytic amounts of acids such as, for example, trifluoroacetic acid, hydrochloric acid, hydrofluoric acid, trifluoromethanesulphonic acid or sulfuric acid. Also suitable is p-toluenesulfonic acid.

For the conversion with the arylamines of the formula (VII), one generally works at temperatures from 20° C. to 80° C., preferably at temperatures of 30° C. to 60° C.

While carrying out the method (C) according to the invention, one adds 0.9 to 1.3 moles, preferably 0.9 to 1.1 moles, particularly preferably 0.9 to 1 moles of the arylamine of the formula (VII) to 1 mole of the isophthalmide of the formula (VI) for creating the phthalic acid diamide of the formula (VI).

The present invention therefore also relates to a method for the production of chiral phthalic acid diamides of the formula (V).

The method (C) according to the invention can be carried out in different method variations, depending on the point in the reaction sequence at which the oxidation step takes place. The following diagram provides two possible variations.

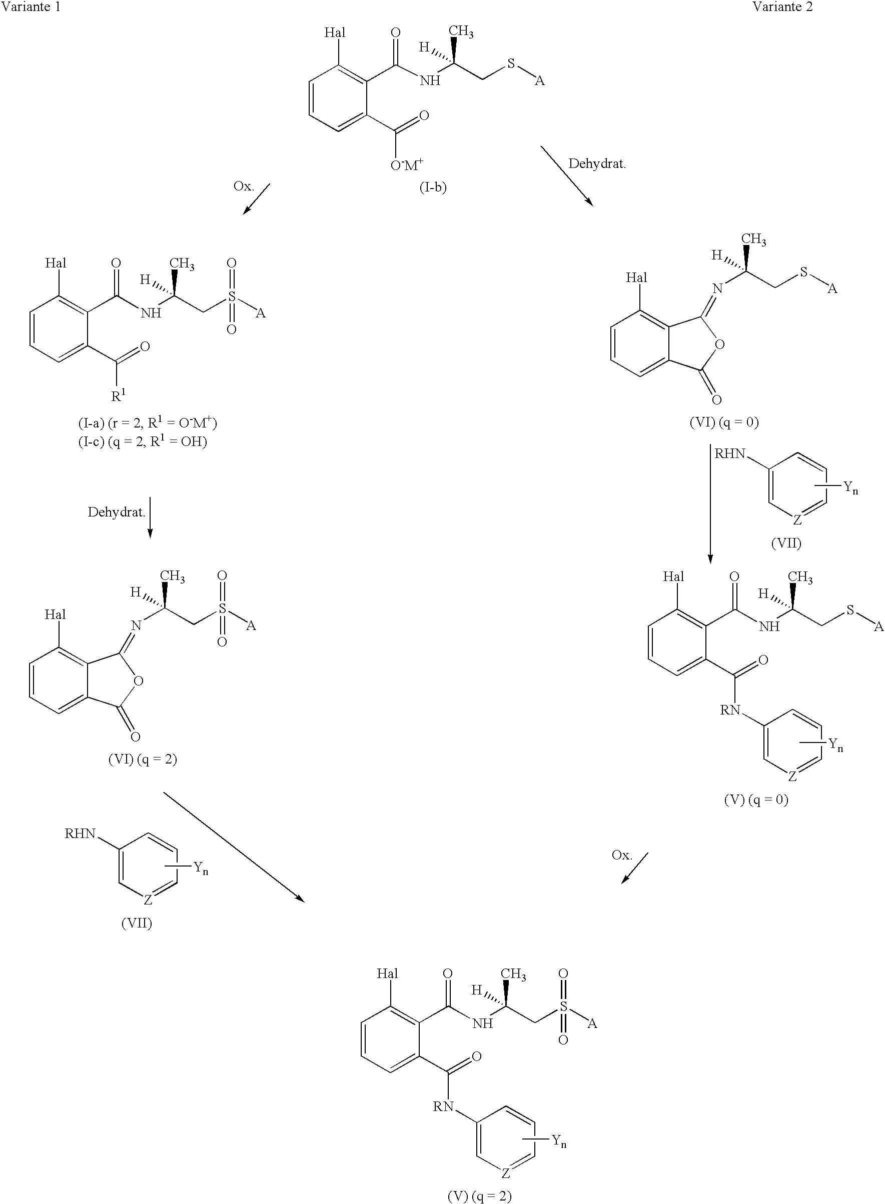

The present invention is illustrated by means of the examples below, which are further illustrated in the scheme above. However, the examples are not be interpreted in a restrictive manner.

### PRODUCTION EXAMPLES

### Example 1

### Synthesis of 3-halo-2-({[(1S)-1-methyl-2-(methylthio)ethyl]amino}carbonyl)benzoates (general method)

One places 0.1 mole of 3-halophthalic acid anhydride in 50 ml of dimethylacetamide and stirs until a homogeneous solution develops. After cooling the reaction mixture to −5° C., one adds 0.1 mole of (2S)-1-(methylthio)propan-2-amine dropwise into dimethylacetamide. The solution is then stirred for 2 hours at room temperature. A solution of lithium hydroxide or sodium hydroxide—depending on which salt one would like to obtain—(0.11 mole) in water is added to the mixture and stirred for 30 minutes at room temperature. The water and the solvent are distilled off in a vacuum. The residue is added to isopropanol, and the suspension is then stirred for 1 hour. The precipitate is filtered and dried.

The compounds listed in the examples 2 to 4 are obtained according to this general description of example 1.

### Example 2

### Lithium-3-brom-2-({[(1S)-1-methyl-2-(methylthio)ethyl]amino}carbonyl)benzoate

Hal stands for bromine, M⁺ stands for lithium.

Yield 82%.

| | |
|---|---|
| ¹H-NMR (CD₃OD): | δ = 1.31(d, 3H), 2.16(s, 3H); 2.42(ddt, 1H); |
| | 2.94(ddt, 1H); 3.30(d, NH); 4.18(dt, 1H); 7.24(1H); |
| | 7.58(1H); 7.76(1H)ppm. |

### Example 3

### Lithium-3-chlor-2-({[(1S)-1-methyl-2-(methylthio)ethyl]amino}carbonyl)benzoate

Hal stands for chlorine, M⁺ stands for lithium.

Yield 88%.

| | |
|---|---|
| ¹H-NMR (CD₃OD): | δ = 1.30(d, 3H); 22.2(s, 3H); 2.43(ddt, 1H); |
| | 2.94(ddt, 1H); 3.30(d, NH); 4.2(dt, 1H); 7.32(1H); |
| | 7.4(1H); 7.7(1H)ppm. |

### Example 4

### Sodium-3-brom-2-({[(1S)-1-methyl-2-(methylthio)ethyl]amino}carbonyl)benzoate

Hal stands for bromine, M⁺ stands for sodium.

Yield 81%.

### Example 5

### Synthesis of 3-halo-2-({[(1S)-1-methyl-2-(methylsulfonyl)ethyl]amino}carbonyl)benzoic acids (general method)

Peracetic acid solution 0.3 mole (as 34% solution in acetic acid) is cooled to 0° C., and 3-halo-2-({[(1S)-1-methyl-2-(methylthio)ethyl]amino}carbonyl)benzoate (0.1 mole) is added in portions. The solution is then stirred for 4 hours at room temperature. The reaction mixture is diluted with water, and the product is extracted with ethyl acetate. The solvent is removed in a vacuum.

The compounds listed in the examples 6 to 8 are obtained according to this general description of example 5.

### Example 6

### 3-iodine-2-({[(1S)1S(methylthio)ethyl]amino}carbonyl)benzoic acid

Benzoate used: Sodium-3-iodine-2-({[(1S)-1-methyl-2-(methylthio)ethyl]amino}carbonyl)benzoate

Yield 85%.

### Example 7

### 3-chloro-2-({[(1S)-1-methyl-2-(methyl sulfonyl)ethyl]amino}carbonyl)benzoic acid

Benzoate used: Lithium-3-chloro-2-({[(1S)-1-methyl-2-methylthio)ethyl]amino}carbonyl)benzoate

Yield 88%.

| | |
|---|---|
| ¹H-NMR (CD₃OD): | δ = 1.31(d, J=6.72 Hz, 3H); 3.16(dd, J=14.47, |
| | 7.16, 1H); 3.45(dd, J=14.47, 5.26 Hz, |
| | 1H); 4.40(s, 1H); 7.53(dd, J=7.89 Hz, |
| | 1H); 7.73(dd, J=8.11, 1.10 Hz, 1H); |
| | 7.88(dd, J=7.82, 1.10 Hz, 1H); 8.56(d, |
| | J=7.89 Hz, 1H). |

### Example 8

### 3-bromo-2-({[(1S)-1-methyl-2-(methylsulfonyl)ethyl]amino}carbonyl)benzoic acid

Benzoate used: Lithium-3-bromo-2-({[(1S)-1-methyl-2-(methylthio)ethyl]amino}carbonyl)benzoate

Yield 84%.

### Example 9

### 3-halo-1-[(1S)-1-methyl-2 (methylthio)ethyl]-N¹-{2-methyl-4-[2,2,2-trifluor-1-(trifluoromethyl)ethyl]phenyl}phthalamide (general method)

Lithium-3-halo-2-({[(1S)-1-methyl-2-(methylthio)ethyl]amino}carbonyl)benzoate (50 mmol) and sodium hydrogen carbonate (83 mmol) are added to 60 ml of water. One adds 30 ml of butyronitrile and heats the reaction mixture to 40° C. After the addition of chloroformic acid methyl ester (74 mmol), it is stirred for 1 hour at 40° C., then cooled to room temperature and the phases separated. The organic phase is dropped into a produced solution of 2-methyl-4-[2,2,2-trifluor-1-(trifluoromethyl)ethyl]aniline (47 mmol) and 0.1 g of p-toluenesulfonic acid in 30 ml of butyronitrile within 30 minutes. The reaction mixture is stirred for 2 hours at 50° C.; the resulting precipitate is filtered and dried.

### Example 10

### 3-halo-N²-[(1S)-1-methyl-2(methylthio)ethyl]-N¹-4-[2,2,2-trifluor-1-(trifluoromethyl)ethyl]phenylphthalamide (general method)

Lithium-3-halo-2-({[(1S)-1-methyl-2-(methylthio)ethyl]amino}carbonyl)benzoate (50 mmol) and sodium hydrogen carbonate (83 mmol) are added to 60 ml of water. One adds 30 ml of butyronitrile and heats the reaction mixture to 40° C. After the addition of chloroformic acid methyl ester (74 mmol), it is stirred for 1 hour at 40° C., then cooled to room temperature and the phases separated. The organic phase is dropped into a produced solution of 2-methyl-4-[2,2,2-trifluor-1-(trifluoromethyl)ethyl]aniline (47 mmol) and 0.1 g of p-toluenesulfonic acid in 30 ml of butyronitrile within 30 minutes. The reaction mixture is stirred for 2 hours at 50° C.; the resulting precipitate is filtered and dried.

### Example 11

### 3-halo-N²-[(1S)-1-methyl-2(methylthio)ethyl]-N¹-{2-methyl-4-[1,2,2-trifluor-1-(trifluoromethyl)ethyl]phenyl}phthalamide (general method)

Lithium-3-halo-2-({[(1S)-1-methyl-2-(methylthio)ethyl]amino}carbonyl)benzoate (50 mmol) and sodium hydrogen carbonate (83 mmol) are added to 60 ml of water. One adds 30 ml of chlorobenzene and heats the reaction mixture to 40° C. After the addition of chloroformic acid methyl ester (74 mmol), it is stirred for 1 hour at 40° C., then cooled to room temperature and the phases separated. The organic phase is dropped into a produced solution of 2-methyl-4-[1,2,2-trifluor-1-(trifluoromethyl)ethyl]aniline (47 mmol), 0.1 g of H₂SO4 in 30 ml of chlorobenzene within 30 minutes. The reaction mixture is stirred for 2 hours at 50° C.; the resulting precipitate is filtered and dried.

### Example 12

### 3-halo-N²-[(1S)-1-methyl-2(methylthio)ethyl]-N¹-[(2-methyl-6-pentafluoroethyl)]pyridyl}phthalamide (general method)

Lithium-3-halo-2-({[(1S)-1-methyl-2-(methylthio)ethyl]amino}carbonyl)benzoate (50 mmol) and sodium hydrogen carbonate (83 mmol) are added to 60 ml of water. One adds 30 ml of butyronitrile and heats the reaction mixture to 40° C. After the addition of chloroformic acid methyl ester (74 mmol), it is stirred for 1 hour at 40° C., then cooled to room temperature and the phases separated. The organic phase is dropped into a produced solution of 2-methyl-3-amino-6-pentafluoroethylpyridine (47 mmol), 0.1 g of toluenesulfonic acid in 30 ml of chlorobenzene within 30 minutes. The reaction mixture is stirred for 2 hours at 50° C.; the resulting precipitate is filtered and dried.

### Example 13

### 3-halo-N²-[(1S)-1-methyl-2(methylthio)ethyl]-N¹-{2-methyl-4-[1,2,2,2-tetrafluor-1-(trifluoromethyl)ethyl]phenyl}phthalamide (general method)

Lithium-3-halo-2-({[(1S)-1-methyl-2-(methylthio)ethyl]amino}carbonyl)benzoate (50 mmol) and sodium hydrogen carbonate (83 mmol) are added to 60 ml of water. One adds 30 ml of butyronitrile and heats the reaction mixture to 40° C. After the addition of chloroformic acid methyl ester (74 mmol), it is stirred for 1 hour at 40° C., then cooled to room temperature and the phases separated. The organic phase is dropped into a produced solution of 2-methyl-4-heptafluoroisopropylaniline (47 mmol) in 30 ml of butyronitrile within 30 minutes. The reaction mixture is stirred for 2 hours at 50° C.; the resulting precipitate is filtered and dried.

The compounds listed in the examples 14 to 20 are obtained according to this general description of example 13.

### Example 14

### 3-bromo-N²-[(1S)-1-methyl-2(methylthio)ethyl]-N¹-{2-methyl-4-[1,2,2,2-tetrafluor-1-(trifluoromethyl)ethyl]phenyl}phthalamide

Starting Materials:

17 g of lithium-3-bromo-2-({[(1S)-1-methyl-2-(methylthio)ethyl]amino}carbonyl)benzoate, 7 g of sodium hydrogen carbonate, 7 g of chloroformic acid methyl ester, 13 g of 2-methyl-4-[1,2,2,2-tetrafluor-1-(trifluoromethyl)ethyl]aniline.

One obtains 25 g of the product (melting point 209-211° C.).

### Example 15

### 3-iodo-N²-[(1S)-1-methyl-2(methylthio)ethyl]-N¹-{2-methyl-4-[1,2,2,2-tetrafluor-1-trifluoromethyl)ethyl]phenyl}phthalamide

Starting Materials:

Lithium-3-iodo-2-({[(1S)-1-methyl-2-(methylthio)ethyl]amino}carbonyl)benzoate.

Yield 87%, melting point 202° C.

### Example 16

### 3-bromo-N²-[(1S)-1-methyl-2(methylthio)ethyl]-N¹-4-[2,2,2-trifluor-1-(trifluoromethyl)ethyl]phenyl}phthalamide

Starting Materials:

17 g of lithium-3-bromo-2-({[(1S)-1-methyl-2-(methylthio)ethyl]amino}carbonyl)benzoate, 7 g of sodium hydrogen carbonate, 7 g of chloroformic acid methyl ester, 13.5 g of 4-[2,2,2-trifluor-1-(trifluor-methyl)ethyl]aniline.

One obtains 24 g of the product.

### Example 17

### 3-chloro-N²-[(1S)-1-methyl-2(methylthio)ethyl]-N¹-{2-methyl-4-[2,2,2-trifluor-1-(trifluoromethyl)ethyl]phenyl}phthalamide

Starting Materials:

Lithium-3-chloro-2-({[(1S)-1-methyl-2-(methylthio)ethyl]amino}carbonyl)benzoate, 2-methyl-4-[2,2,2-trifluor-1-(trifluoromethyl)ethyl]aniline

Yield 88%. Melting point 190-195° C.

### Example 18

### 3-chloro-N²-[(1S)-1-methyl-2(methylthio)ethyl]-N¹-{2-methyl-4-[1,2,2-trifluor-1-(trifluoromethyl)ethyl]phenyl}phthalamide

Starting Materials:

Lithium-3-chloro-2-({[(1S)-1-methyl-2-(methylthio)ethyl]amino}carbonyl)benzoate, 2-methyl-4-[1,2,2-trifluor-1-(trifluoromethyl)ethyl]aniline

Yield 84%, melting point 185-188° C.

### Example 19

### 3-chloro-N²-[(1S)-1-methyl-2(methylthio)ethyl]-N¹-{2-methyl-4-[1,2,2,2-tetrafluor-1-(trifluoromethyl)ethyl]phenyl}phthalamide

Starting Materials:

Lithium-3-chloro-2-({[(1S)-1-methyl-2-(methylthio)ethyl]amino}carbonyl)benzoate, 2-methyl-4-[1,2,2,2-tetrafluor-1-(trifluoromethyl)ethyl]aniline

Yield 84%, melting point 207-208° C.

### Example 20

### 3-iodo-N²-[(1S)-1-methyl-2-(methylthio)ethyl]-N¹-{2-methyl-4-[2,2,2-trifluor-1-(trifluoromethyl)ethyl]phenyl}phthalamide

Starting Materials:

Lithium-3-iodo-2-({[(1S)-1-methyl-2-(methylthio)ethyl]amino}carbonyl)benzoate, 2-methyl-4-[2,2,2-trifluor-1-(trifluoromethyl)ethyl]aniline

Yield 88%.

### Example 21

### 3-bromo-N²-[(1S)-1-methyl-2(methylsulfonyl)ethyl]-N¹-{2-methyl-4-[1,2,2,2-tetrafluor-1-(trifluoromethyl)ethyl]phenyl}phthalamide

50 mmol of 3-bromo-N²-[(1S)-1-methyl-2(methylthio)ethyl]-N¹-{2-methyl-4-[1,2,2,2-tetrafluor-1-(trifluoromethyl)ethyl]phenyl}phthalamide is suspended in 80 ml of chlorobenzene and added to 20 ml of hydrogen peroxide. The mixture is stirred for 1 hour at 45° C. and then cooled to room temperature. The precipitate is filtered and washed with cold chlorobenzene. Yield: 92%.

### Example 22

### (3Z/E)-4-halo-3-({[(1S)-1-methyl-2-(methylthio)ethyl]imino}2-benzofuran-1(3H)-one (general method)

Lithium-3-halo-2-({[(1S)-1-methyl-2-(methylthio)ethyl]amino}carbonyl)benzoate (50 mmol) and sodium hydrogen carbonate (83 mmol) are placed in 60 ml of water. One adds 30 ml of butyronitrile and heats the reaction mixture to 40° C. After the addition of chloroformic acid methyl ester (74 mmol), it is stirred for 1 hour at 40° C., then cooled to room temperature and the phases separated. The organic phase is concentrated.

The compounds listed in the examples 23 to 24 are obtained according to this general description of example 22.

### Example 23

### (3Z/E)-4-iodo-3-({[(1S)-1-methyl-2-(methylthio)ethyl]imino}2-benzofuran-1(3H)-one

Starting Materials:

Lithium-3-iodo-2-({[(1S)-1-methyl-2-(methylthio)ethyl]amino}carbonyl)benzoate.

Yield: 91%, melting point 88-89° C.

### Example 24

### (3Z/E)-4-halo-3-({[(1S)-1-methyl-2-(methylthio)ethyl]imino}2-benzofuran-1(3H)-one

Starting Materials:

Lithium-3-chloro-2-({[(1S)-1-methyl-2-(methylthio)ethyl]amino}carbonyl)benzoate.

Yield: 93%, melting point 71-72° C.

### Example 25

### (3Z/E)-4-chloro-3-{[(1S)-1-methyl-2-(methylthio)ethyl]imino}2-benzofuran-1(3H)-one

Lithium-3-chloro-2-({[(1S)-1-methyl-2-(methylthio)ethyl]amino}carbonyl)benzoate (50 mmol), sodium hydrogen carbonate (83 mmol) and tetrabutylammonium hydrogen sulfate (0.5 mmol) are placed in 40 ml of water. One adds 40 ml of monochlorobenzene and then (60 mmol) chloroformic acid methyl ester. One stirs the reaction mixture for 3 hours at room temperature. The organic phase is separated and concentrated in a vacuum. One obtains 47 mmol (95% of the theory) of the product with 98% purity.

1. Method for the production of 3-halophthalic acid derivatives of the formula (I) 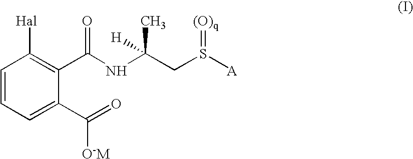
in which
Hal stands for halogen,
A stands for C₁-C₆-alkyl, C₃-C₆-alkenyl, C₃-C₆-alkinyl, C₁-C₆-alkoxy-C₁-C₄-alkyl, C₁-C₆-alkylthio-C₁-C₄-alkyl, C₁-C₆-alkylsulfinyl-C₁-C₄-alkyl, (C₁-C₆-alkyl)carbamoyl,
q stands for 0, 1 or 2,
M stands for an alkali metal ion, an earth alkali metal ion, tetra(C₁-C₄-alkyl)ammonium or tetra(C₁-C₄-alkyl)phosphonium, whereby in the case of an earth alkali metal ion respectively two molecules of a compound of the formula (I) form a salt with one such ion,
characterised in that one causes to react
(A) 3-halophthalic acid anhydrides of the formula (II) 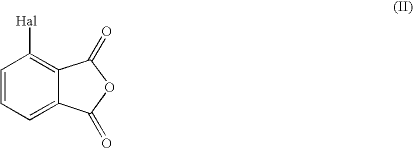
in which Hal has the meaning indicated above,
with amines of the formula (III) 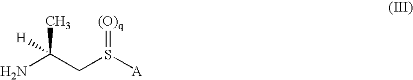
in which A and q have the meanings indicated above,
in the presence of a hydroxide of the formula (IV)
M(OH⁻)ₜ (IV)
in which
M has the meaning indicated above,
t stands for 1, if M stands for alkali metal ion, tetra(C₁-C₄-alkyl)ammonium or tetra(C₁-C₄-alkyl)phosphonium.
t stands for 2 if M stands for an earth alkali metal ion.

2. Method according to claim 1, characterised in that
Hal stands for fluorine, chlorine, bromine or iodine,
A stands for methyl, ethyl, n- or iso-propyl, n-, iso-, sec- or tert-butyl, allyl, propargyl, methoxymethyl or methylthiomethyl,
M stands for lithium, sodium, potassium, calcium, magnesium, barium, tetrabutylammonium or tetrabutylphosphonium.

3. Method according to claim 1, characterised in that
Hal stands for fluorine, chlorine, bromine or iodine,
A stands for methyl, ethyl, n- or iso-propyl, methoxymethyl or methylthiomethyl,
q stands for 0,
M stands for lithium or sodium.

4. Method according to claim 1, characterised in that
Hal stands for chlorine, bromine or iodine,
A stands for methyl, ethyl, n- or iso-propyl,
q stands for 0,
M stands for lithium.

5. Method according to claim 1, characterised in that
Hal stands for iodine, chlorine or bromine,
A stands for C₁-C₆-alkyl,
q stands for 0,
M stands for lithium, sodium or potassium.

6. Method according to claim 1, characterised in that one first obtains compounds of the formula (I), in which q stands for 0, and then oxidises them to compounds of the formula (I-a) 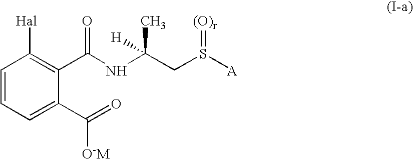
in which
r stands for 1 or 2 and
Hal, A and M have the meanings indicated above in claim 1.

7. Isophthalimides of the formula (IV) 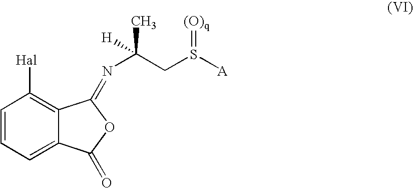
in which
Hal stands for halogen,
A stands for C₁-C₆-alkyl, C₃-C₆-alkenyl, C₃-C₆-alkinyl, C₁-C₆-alkoxy-C₁-C₄-alkyl, C₁-C₆-alkylthio-C₁-C₄-alkyl, C₁-C₆-alkylsulfinyl-C₁-C₄-alkyl, (C₁-C₆-alkyl)carbamoyl,
q stands for 0, 1 or 2.

8. 3-Halophthalic acid derivatives of the formula (I) 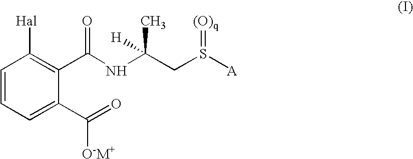
in which
Hal stands for halogen,
A stands for C₁-C₆-alkyl, C₃-C₆-alkenyl, C₃-C₆-alkinyl, C₁-C₆-alkoxy-C₁-C₄-alkyl, C₁-C₆-alkylthio-C₁-C₄-alkyl, C₁-C₆-alkylsulfinyl-C₁-C₄-alkyl, (C₁-C₆-alkyl)carbamoyl,
q stands for 0, 1 or 2,
M stands for an alkali metal ion, an earth alkali metal ion, tetra(C₁-C₄-alkyl)ammonium or tetra(C₁-C₄-alkyl)phosphonium, whereby in the case of an earth alkali metal ion respectively two molecules of a compound of the formula (I) form a salt with one such ion,

9. 3-Halophthalic acid derivatives of the formula (I) according to claim 8, in which
Hal stands for fluorine, chlorine, bromine or iodine,
A stands for methyl, ethyl, n- or iso-propyl, n-, iso-, sec- or tert-butyl, allyl, propargyl, methoxymethyl or methylthiomethyl,
M stands for lithium, sodium, potassium, calcium, magnesium, barium, tetrabutylammonium or tetrabutylphosphonium.

10. 3-Halophthalic acid derivatives of the formula (I) according to claim 8, in which
Hal stands for chlorine, bromine or iodine,
q stands for 0,
M stands for lithium or sodium.

11. 3-Halophthalic acid derivatives of the formula (I-c) 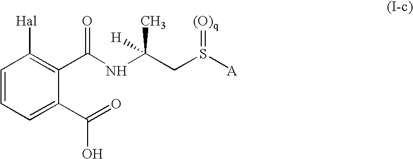
in which
Hal stands for halogen,
A stands for C₁-C₆-alkyl, C₃-C₆-alkenyl, C₃-C₆-alkinyl, C₁-C₆-alkoxy-C₁-C₄-alkyl, C₁-C₆-alkylthio-C₁-C₄-alkyl, C₁-C₆-alkylsulfinyl-C₁-C₄-alkyl, (C₁-C₆-alkyl)carbamoyl,
q stands for 0, 1 or 2,

12. Use of 3-halophthalic acid derivatives of the formula (I) according to claim 8 for the production of phthalic acid diamides of the formula (V) 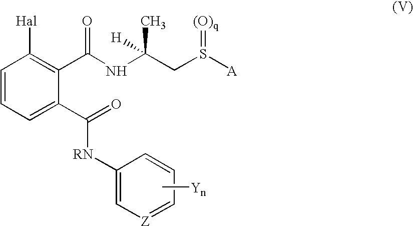
in which
Hal stands for halogen,
A stands for C₁-C₆-alkyl, C₃-C₆-alkenyl, C₃-C₆-alkinyl, C₁-C₆-alkoxy-C₁-C₄-alkyl, C₁-C₆-alkylthio-C₁-C₄-alkyl, C₁-C₆-alkylsulfinyl-C₁-C₄-alkyl, (C₁-C₆-alkyl)carbamoyl,
q stands for 0, 1 or 2,
R stands for hydrogen or C₁-C₆ alkyl,
Z stands for CY or N,
Y stands for hydrogen, halogen, C₁-C₆ alkyl, C₁-C₆-halogenoalkyl, C₁-C₆-alkoxy, C₁-C₆-halogenoalkoxy, C₁-C₆-alkylthio, C₁-C₆-halogenoalkylthio or cyano,
n stands for 0, 1, 2, 3, 4 or 5,

13. Method for the production of phthalic acid diamides of the formula (V) 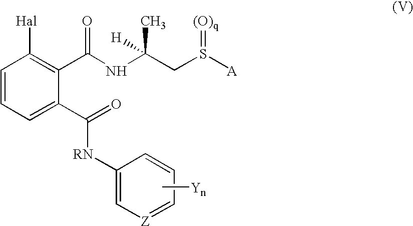
in which
Hal stands for halogen,
A stands for C₁-C₆-alkyl, C₃-C₆-alkenyl, C₃-C₆-alkinyl, C₁-C₆-alkoxy-C₁-C₄-alkyl, C₁-C₆-alkylthio-C₁-C₄-alkyl, C₁-C₆-alkylsulfinyl-C₁-C₄-alkyl, (C₁-C₆-alkyl)carbamoyl,
q stands for 0, 1 or 2,
R stands for hydrogen or C₁-C₆ alkyl,
Z stands for CY or N,
Y stands for hydrogen, halogen, C₁-C₆ alkyl, C₁-C₆-halogenoalkyl, C₁-C₆-alkoxy, C₁-C₆-halogenoalkoxy, C₁-C₆-alkylthio, C₁-C₆-halogenoalkylthio or cyano,
n stands for 0, 1, 2, 3, 4 or 5,
characterised in that one causes to react
(C) 3-halophthalic acid derivatives of the formula (I) 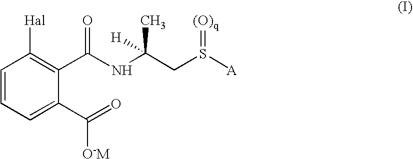
in which Hal, A, q and M have the meanings indicated above,
first with a dehydrating reagent in the corresponding isophthalimides of the formula (VI) 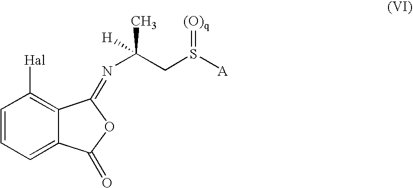
in which Hal, A and q have the meanings indicated above,
and causes them to react after isolation or without further isolation with arylamines of the formula (VII) 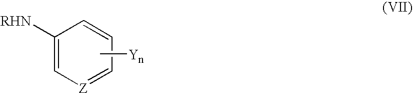
in which R, Z, Y and n have the meanings indicated above,
if necessary in the presence of a diluent (for example, chlorobenzene) and if necessary in the presence of an acid (for example, hydrochloric acid).

14. Method according to claim 13, characterised in that one converts the 3-halophthalic acid derivatives of the formula (I) to the isophthalimides of the formula (VI) in the presence of a phase transfer catalyst.

15. Method according to claim 13, characterised in that one selects the phase transfer catalyst from the series tetramethylammonium bromide, tetrabutylammonium hydroxide, tetrabutylammonium hydrogen sulfate, tetraphenylphosphonium bromide, 18-crown-6.

16. Method according to claim 13, characterised in that on uses tetrabutylammonium hydrogen sulfate as a phase transfer catalyst.

17. Method according to the claims 13-16, characterised in that the synthesis of compounds of the formula (V) is carried out as a one-pot reaction without isolation of intermediate products.
